(19) United States
(12) Patent Application Publication
(10) Pub. No.: US 2022/0160648 A1
(43) Pub. Date: May 26, 2022

(54) Composition Comprising Hydrolyzed Collagen and Cannabidiol and Use Thereof

(71) Applicant: Jim Caras, Irvine, CA (US)

(72) Inventor: Jim Caras, Irvine, CA (US)

(73) Assignee: Inspired Health Products, LLC, Irvine, CA (US)

(21) Appl. No.: 17/430,803

(22) PCT Filed: Feb. 13, 2020
(86) PCT No.: PCT/US2020/018105

§ 371 Date: Aug. 13, 2021

Related U.S. Application Data
(60) Provisional application No. 62/805,067, filed on Feb. 13, 2019.

(51) Int. Cl. A61K 31/05; A61K 38/39; A61K 31/01; A61K 31/015; A61K 31/045; A61K 9/00; A61P 25/20

(52) U.S. Cl. CPC A61K 31/05; A61K 38/39; A61K 31/01; A61P 25/20; A61K 31/045; A61K 9/0053; A61K 31/015

(57) ABSTRACT

Composition comprising enzymatically hydrolyzed collagen and cannabidiol, the composition suitable for ingestion by an individual to facilitate improvements in rest, including sleep, and/or in recovery, including but not limited to recovery after physical and/or mental exertion, recovery from pain and combinations thereof, and to facilitate improved delivery to the individual of amino acids comprised in the enzymatically hydrolyzed collagen. Both liquid and particulate compositions comprising full spectrum cannabidiol oil or cannabidiol isolate are provided as well as compositions comprising one or more of a terpene isolate, flavonoid, essential oil, effervescent agent, sweetener or flavoring agent.

### CROSS-REFERENCE TO RELATED APPLICATION

The present application claims the benefit of the filing date of U.S. Provisional Patent Application No. 62/805,067, filed Feb. 13, 2019, the disclosure of which is hereby incorporated herein by reference.

### BACKGROUND OF THE INVENTION

Cannabidiol (shorthand reference CBD) is a phytocannabinoid discovered in 1940. Based on naming rules for chemical compounds according to the International Union of Pure and Applied Chemistry (IUPAC), the chemical compound can be identified as 2-[(1R,6R)-3-methyl-6-prop-1-en-2-ylcyclohex-2-en-1-yl]-5-pentylbenzene-1,3-diol or 2-[1R-3-methyl-6R-(1-methylethenyl)-2-cyclohexen-1-yl]-5-pentyl-1,3-benzenediol.

The CBD molecule is understood to exist in the form of two molecular configurations in the asymmetric unit, differing mainly in the n-pentyl side-chain conformation (Cannabidiol, P. G. Jones et al., Acta Cryst. (1977), B33, 3211-3214). CBD is normally taken to refer to the naturally occurring (−)-enantiomer. The (+) CBD enantiomer has been synthesized. The simplified chemical structure of both CBD configurations is shown below; for purposes of the present invention both configurations are equivalent and may be present in CBD useful herein. Properties described herein are attributable to the presence of both configurations in CBD.

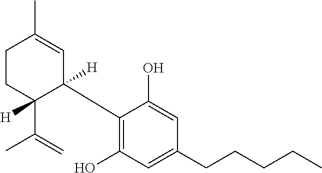 [SMILES: C=C(C)[C@@H]1CCC(C)=C[C@H]1c1c(O)cc(CCCCC)cc1O]

CBD is said to be one of approximately 113 cannabinoids identified in hemp plants, accounting for up to 40% of the plant's extract. As of 2018, CBD is considered to have no psychoactive properties or psychotropic effects in people such as those caused by Δ9-tetrahydrocannabinol (THC), also identified as (6aR,10aR)-delta-9-tetrahydrocannabinol, which is found in marijuana. CBD has reportedly been used for various medical purposes, including treatment for certain types of childhood epilepsy and it may also have possible therapeutic uses for the treatment of various neurological disorders, although initial findings have not been confirmed by high-quality clinical research normally required to establish such uses in clinical practice.

CBD may have anti-anxiety and anti-psychotic effects based on animal studies, which indicate it has potential as an anxiolytic for relief of anxiety-related disorders and fear. Preliminary research also shows that CBD may have potential for improving addictive disorders and drug dependence, although evidence for such effect in people is limited. CBD is also reported to have an anti-inflammatory effect. (Proc. Natl. Acad. Sci. USA (PNAS), Aug. 15, 2000, vol. 97, no. 17, 9561-9566) Thus, there is a desire for improved compositions comprising CBD for human and animal consumption.

Hydrolyzed collagen (HC) is referred to by alternative names, which, for the purposes of the present application are to be considered equivalent, and referred to simply as hydrolyzed collagen including, for example, gelatin or gelatine, collagen hydrolysate, gelatin hydrolysate, hydrolyzed gelatin, collagen peptides, hydrolyzed collagen peptides and enzymatically hydrolyzed collagen. Hydrolyzed collagen is typically derived from collagen obtained from various animal body parts. Collagen has also been produced via tobacco plant-based expression of recombinant human collagen.

When hydrolyzed, collagen is reduced to smaller peptides, which can be ingested in compositions in the form of a dietary supplement or functional foods and beverages with the potential to aid joint and bone health and enhance skin health and increase lean body mass as well as for other purposes. Hydrolyzed collagen has a much smaller or lower molecular weight in comparison to native or unhydrolyzed collagen or gelatin, and study suggests that more than 90% of hydrolyzed collagen is digested and available as small peptides in the blood stream within one hour. From the blood, the peptides (containing hydroxyproline) are transported into target tissues, including, for example skin, bones, and cartilage, where the peptides act as building blocks for local cells and help boost the production of new collagen fibers.

Hydrolyzed collagen can function as a scaffolding material for regeneration and healing in human and animal applications. Collagen is an abundant macromolecule of the extracellular matrix and connective tissue, and it is intimately involved in tissue development, remodeling and repair. Collagen-based products, including hydrolyzed collagen, are generally recognized as safe for application, injection, implantation and oral ingestion and collagen as well as hydrolyzed collagen has served as a central element in a myriad of medical and cosmetic products, which harness its biological role in tissue structure and repair processes. Its benefits have been exploited in the design of biomaterials such as wound dressings, dermal patches and fillers, bone and tendon substitutes, and engineered tissues, which can be impregnated with exogenous growth factors, drugs, and/or cells.

Improved hydrolyzed collagen-containing compositions comprising cannabidiol are desirable, advanced compositions especially for human applications.

### SUMMARY OF THE INVENTION

A composition comprising hydrolyzed collagen, preferably enzymatically hydrolyzed collagen and cannabidiol, such as full spectrum cannabidiol oil or cannabidiol isolate. Hydrolyzed collagen and cannabidiol compositions comprising additional components selected from at least one terpene isolate, at least one essential oil are also disclosed. Disclosed compositions facilitate improvements in rest, including sleep, as well as in recovery, including but not limited to recovery after physical or mental exertion, recovery from pain and combinations thereof, of an individual in need thereof.

### DETAILED DESCRIPTION

The present invention is directed to compositions comprising defined components including hydrolyzed collagen or gelatin hydrolysate, and cannabidiol (CBD) as a component of CBD compositions, such as CBD oil, CBD in substantially pure form or CBD in admixture with other liquid or solid components. Compositional embodiments include liquids, liquid mixtures and liquid dispersions as well as solids or powder, for example particulate, mixtures; the components can be present in a mixture, or a dispersion or emulsion, particularly where a liquid or aqueous component or carrier is present.

It is understood that CBD is oil soluble and it can be provided in the form of “CBD oil”, which is described hereinbelow, whereas hydrolyzed collagen is typically soluble or substantially soluble in water. Although an aqueous composition comprising hydrolyzed collagen, especially partially hydrolyzed collagen, may be subject to gelation when cooled, an aqueous composition comprising enzymatically hydrolyzed collagen typically does not gel. Hydrolyzed collagen is also sometimes referred to as collagen peptides or hydrolyzed peptides as a consequence of hydrolysis carried out on the collagen starting material. Collagen peptides or hydrolyzed collagen, contain the same amino acids as the original unhydrolyzed collagen, but hydrolyzed collagen exhibits a lower molecular weight than the starting material. Since collagen peptides are lower in molecular weight they can be absorbed more efficiently, for example more quickly, into the bloodstream than the original long chain, high molecular weight peptides.

When desired, compositions of the invention comprise optional ingredients including, for example, one or more sweeteners, such as sorbitol, a palatable acid, such as citric acid, fumaric acid or adipic acid, one or more isolated amino acids, such as tryptophan, a synthetic sweetener, flavoring agents, preservatives and/or stabilizers, emulsifiers or surfactants for aqueous compositions or compositions which are to be combined with water, as well as effervescent components, particularly in compositions comprising hydrolyzed collagen powder and CBD in powder or crystalline form, for example in the form of particulates. The flavoring agent impart a neutral or other flavor including one which is highly palatable and sweet, tart or sour, for example, cherry, orange, green apple, or the like, while the preservative agents may be any one or more of a number of preservatives generally recognized as safe for human consumption, such as potassium sorbate, sodium benzoate, and the methyl-, propyl-, butyl-, and ethylesters of p-hyroxybenzoic acid, the latter esters available under trade names methyl paraben, propyl paraben, etc. Preservatives can be used either alone or in admixture.

2) Hydrolyzed Collagen

Collagen is the major insoluble fibrous protein in the extracellular matrix and in connective tissue; it is the single most abundant protein in the animal kingdom. There are at least 16 types of collagen, and 80 to 90% of the collagen in the mammalian body typically consists of types I, II, and III. Collagen molecules pack together to form long thin fibrils of similar structure.

Hydrolyzed collagen can be made by hydrolyzing animal collagen. For example, animal collagen can be derived from the skin and/or bones and/or connective tissue of one or more animals selected from pig, bovine, ox, cow, calf, bull, sheep, goat, antelope and buffalo as well as from fish and chicken. Preferred are commercially available hydrolyzed collagen products derived, for example, from the skin of pork bellies (porcine collagen) and the cartilage, bones and hides of cattle, e.g. cows, calves, etc. (bovine collagen), by means of well-known hydrolysis and extraction processes to produce hydrolyzed collagen. Bovine or porcine collagen comprises Type I and Type III collagen. On the other hand, marine collagen, such as collagen-rich fish skin and bones from ocean fish such as cod, haddock and Pollock, among others, is typically a large portion of the estimated 60% of fish by-products. On the other hand, marine collagen consists primarily of Type I collagen. Chicken derived collagen is primarily extracted from chicken breast cartilage and it is typically comprised of Type II collagen.

The amino acid content (types and amounts) in hydrolyzed collagen are the same as in the collagen from which it is prepared. Hydrolyzed collagen contains 19 amino acids, predominantly glycine, proline and hydroxyproline, which together represent around 50% of the total amino acid content. Typical amino acid distribution in collagen and hydrolyzed collagen is shown in the following table.

| Amino acids | Percentage |
|---|---|
| Proline and hydroxyproline | 25% |
| Glycine | 20% |
| Glutamic acid | 11% |
| Arginine | 8% |
| Alanine | 8% |
| Other essential amino acids | 16% |
| Other non-essential amino acids | 12% |

Hydrolyzed collagen contains 8 out of 9 essential amino acids, including glycine and arginine—two amino-acid precursors necessary for the biosynthesis of creatine. It contains no tryptophan and is deficient in isoleucine, threonine, and methionine.

Native, natural or unhydrolyzed collagen has a molecular weight in the range of about 300-500 kDa. Following hydrolysis, the hydrolyzed collagen exhibits a molecular weight of about 0.3 to 8 kDa, depending on the original collagen, the hydrolysis method and the extent of hydrolysis carried out. Hydrolyzed collagen, including enzymatically hydrolyzed collagen useful in the present invention can have a molecular weight within the typical range noted above; preferably, hydrolyzed collagen will exhibit a molecular weight in the range of about 1 kDa to 7 kDa, or about 2 kDa to about 6 kDA, more preferably about 5 kDa or less, for example, about 2 kDA to about 6 kDa, such as 2 kDa, or 3 kDa or 4 kDa or 5 kDa, or 6 kDa.

Various of the above-described types of collagen, alone or in combination, can be useful for producing hydrolyzed collagen for use in compositions and methods of the present invention, although collagen Types I and III are preferred; more preferred are Types I and III derived from porcine and bovine sources; most preferred is bovine collagen; especially preferred are hydrolyzed, most especially enzymatically hydrolyzed porcine and/or bovine collagen.

Hydrolyzed collagen is available commercially and can be obtained from various manufacturers including, e.g., Gelita, Nitta Gelatin, Ewald Gelatine, Reinert Gruppe Ingredients, PB Leiner, Jellice and Trobas Gelatine, Gelatines Weishardt, Junca Gelatines and Rousselot, Inc., Foodmate Co., Ltd., as well as others worldwide.

If collagen is partially hydrolyzed, for example, using acid or base processes known in the art, the resulting product can be soluble in warm water, but upon cooling may result in a gel. In contrast, an aqueous composition of enzymatically hydrolyzed collagen does not gel. It is particularly preferred to carry out enzymatic hydrolysis rather than acid or base hydrolysis because the use of an enzyme converts collagen to more palatable small peptides (i.e., mono-, di-, or tri-peptides) rather than to the less palatable amino acids. In addition, enzymatic hydrolysis produces fewer distasteful impurities. Furthermore, if acid or base hydrolysis is used it can be further enhanced by enzymatic hydrolysis to produce the final, desired hydrolyzed collagen product useful herein.

The structural breakdown of proteins, for example by enzymatic hydrolysis, is also referred to as proteolysis. Similarly, a proteolytic enzyme that weakens or breaks the peptide linkages in proteins is referred to as a protease. Many food grade proteases are available for protein hydrolysis and they can be characterized by their origin, e.g., animal, plant or microbial as well as their mode of action. For example, endoproteases cleave amide bonds within the protein chain and exoproteases remove terminal amino acids from proteins or peptides. Examples of proteases useful for hydrolysis of food proteins and potentially useful herein include the serine proteases trypsin, chymotrypsin, and elastase; and the bacterially sourced Bacillus licheniformis (commercially available as “Alcalase”) and amyloliquefaciens (e.g., “Substilsin Novo”). Cysteine proteases from plants include papain, bromelain and ficin. Aspartic proteases from animals include pepsin (from porcine and bovine sources) and chymosin (from calves). Fungal aspartic proteases are considered chymosin-like (from Mucor pusillus and miehei or endothia parasitica), aspergillo-peptidase A and newlase (from rhizopus sp.). Animal metallo protease such as carboxy peptidase A (from the pancreas) and bacterial metallo proteases such as neutral protease (commercially available as “Neutrase” and “Thermolysin”) from Bacillus amyloliquefaciens and thermoproteolyticus, respectively. Commercial mixtures of proteases are available such as crude papain, which is a mixture of papain, chymopapain, and lysozyme; pancreatin, which is a mixture of trypsin, chymotrypsin, elastase, and carboxypeptidase; “Veron P”, “Sumyzyme LP”, and “Biozyme A,” which are mixtures of serine-, aspartic-, and metalloprotease; and “Pronase,” which is a mixture of endo- and exoproteases, active at neutral and alkaline pH. A compilation of useful food-grade proteases is published in the PhD dissertation by C. van der Ven, entitled Biochemical and functional characterization of casein and whey protein hydrolysates.” Jun. 4, 2002. Preferred enzymes for use in the hydrolysis of collagen are those generally recognized as safe for human consumption. The enzymes particularly preferred are bromelain, papain, and ficin, especially papain, although other enzymes described herein may also be used. Processes for hydrolysis, including enzymatic hydrolysis of the collagen protein sources identified above and useful in the present invention are well known to those skilled in that art.

For convenience, hydrolyzed collagen compositions can also be described in terms of amino acids present in the composition. Collagen is especially rich in the amino acid glycine, and it is the only protein known to contain a substantial proportion of hydroxyproline. However, hydrolyzed collagen, including enzymatically hydrolyzed collagen based on animal collagen, does not contain the essential amino acid tryptophan. Therefore, where all essential amino acids may be desired to be in the composition, tryptophan can be added to compositions useful in the present invention in an effective amount, for example from about 0.02 to about 2.0 parts by weight of the composition. Other adjustments in the overall amino acid content as well as for individual amino acid components may also be made as desired or necessary or for special purposes or applications. For example, the individual and overall amount of amino acids used should be such that an effective amount of each is provided even though the hydrolyzed collagen source from which the amino acids is obtained varies, e.g., in moisture content. Additionally, if desired, the composition can include an additional amount of one or more amino acid beyond that naturally present in the hydrolyzed animal collagen. For example, an additional amount of hydrolyzed arginine can be included. The content of each of the amino acids may vary somewhat from batch-to-batch within acceptable values for such a composition. Furthermore, the amino acid profile of a composition suitable for use in the present invention can be varied by as much as ±30% by weight; more typically ±25% by weight; for example, ±20% by weight; provided that, if the amount of any particular amino acid is less than the preferred amount, it is at least sufficient so that the composition is suitable to achieve the desired benefit of the composition.

Compositions of the present invention include a high protein concentration, typically about 250 mg to about 20 grams of essential and non-essential amino acids and combinations thereof: the non-essential amino acids alanine, arginine (also considered an essential amino acid for children), aspartic acid, cystine, glutamic acid, glycine, hydroxylysine, hyroxyproline, proline, serine, and tyrosine; and the essential amino acids histidine, isoleucine, leucine, lysine, methionine, phenylalanine, threonine, tryptophan and valine.

Compositions of the present invention are also useful for maintaining a positive nitrogen balance. Nitrogen balance indicates that the rate of protein synthesis in the body equals that of protein breakdown. It is important to maintain a positive nitrogen balance in the body, in order to preserve muscle tissue and lean body mass. If consumption of protein in one's diet is inadequate, a negative nitrogen balance will result. Subsequently, the body breaks down the protein in its own muscle tissues in order to reverse the imbalance. Protein is the body's main source of nitrogen, and when it breaks down, nitrogen is excreted. Measuring the amount of nitrogen excretion reflects how much protein is breaking down. A negative nitrogen balance indicates wasting away of muscles. It is especially desirable to prevent this.

Following hydrolysis, filtering and purification, hydrolyzed collagen, including enzymatically hydrolyzed collagen, can be concentrated and subjected to drying by methods well-known in the art, including for example, spray drying. The resulting dried, hydrolyzed collagen is conveniently in the form of a powder or particulate.

Cannabidiol

Synthesis of CBD

CBD is available from naturally occurring plant materials, but it can also be synthesized. An efficient synthesis is the acid condensation of p-mentha-2,8-dien-1-ol with olivetol (Cannabidiol: an overview of some chemical and pharmacological aspects. Part I: chemical aspects, R. Mechoulam et al., Chemistry and Physics of Lipids 121 (2002) 35-43). The yield reported (41% of crystalline material) in this one step reaction makes CBD readily available. The reaction scheme below illustrates the synthesis; the first compound illustrated in the reaction scheme is CBD, the other two being co-produced reaction by-products.

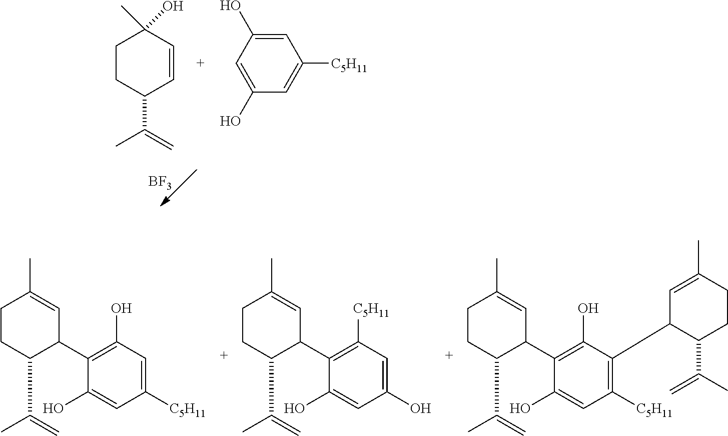

Extraction of CBD

Alternatively, and more commonly, CBD is extracted from natural plant materials. For example, CBD can be extracted from marijuana or industrial hemp plants. Typically, CBD is extracted from the stalks and stems of industrial hemp plants which are cannabis plants containing 0.3% THC or less, which classifies them as “industrial hemp”. The concentration of CBD present in hemp plant material is variable, but nevertheless is readily obtained or extracted from this source material, industrial hemp, by process steps known to those skilled in the art.

Extraction to obtain CBD or CBD cannabis oil starts with CBD-containing plant material that is dried and pulverized followed by extraction. Various known extraction methods are available, including but not limited to:
CO₂ extraction: The supercritical (or subcritical) CO₂ method uses carbon dioxide under high pressure and low temperatures to isolate, preserve, and maintain the purity of extracted CBD and CBD oil. Use of CO₂ at elevated pressure for extraction of desirable components from plants and other materials is generally well-known in the art.
Ethanol extraction: Purified grain alcohol can be used to produce CBD and CBD oil. This extraction method also removes ethanol soluble plant waxes.
Olive oil extraction: Extra virgin or otherwise, olive oil can also be used to extract CBD and CBD oil. This method is safe and inexpensive, however, CBD-infused olive oil is perishable and is typically stored under cool, dark conditions to avoid degradation associated with the presence of the olive oil.

CBD can be dissolved in pentane (as well as other organic solvents, including ethanol, methanol, dimethyl sulfoxide or DMSO, dimethyl formamide and others) and crystallized from the solution. The crystalline melting point of CBD is reportedly 66-67° C., although melting point values have also been reported as 62-63° C. Thus, CBD in crystalline form can be reduced to a powdered or particulate form, provided that temperature is controlled at less than its melting point; CBD powder or CBD in particulate form is available commercially.

Commercially sourced CBD may be in the form of essentially pure CBD, for example ≥99% pure CBD, also referred to as CBD “isolate”, or in the form of CBD containing minor or trace amounts of compounds originally present in the plant from which the CBD was extracted, typically industrial hemp, although CBD can also be extracted from the marijuana plant. CBD containing limited or trace amounts of compounds originally present in industrial hemp in particular is sometimes referred to as “full spectrum” CBD. Trace or limited concentration compounds can include other cannabinoids, plant resin and or oil, phytocannabinoids and plant terpenes, flavonoids and other trace compounds including B complex vitamins, omega-3 and omega-6 fatty acids, essential amino acids, vitamins, including vitamins E, A and C, and minerals, including magnesium, potassium, iron, calcium, zine and phosphorus. Pure CBD, also referred to as CBD isolate, and full spectrum CBD are useful herein and may be referred to conveniently as CBD.

Conversion of CBD, especially CBD isolate or crystalline CBD to a powder can be accomplished by methods well-known to those skilled in the art using for example a mortar and pestle for small amounts or a powder mill for larger amounts. As noted above, temperature control is important in order to avoid melting so as to preserve a powder form. Particle size can be selected as convenient for admixture with hydrolyzed or enzymatically hydrolyzed collagen and other components where desired.

Cannabidiol Oil

For purposes of the present invention, cannabidiol (CBD) oil is essentially a concentrated extract, for example a solvent extract, preferably made from industrial hemp (although it can also be produced from cannabis flowers and/or leaves) that is dispersed, preferably dissolved, in an edible oil such as sunflower, hemp, or olive oil. Solvents used for extraction can vary from organic alcohols such as ethanol and isopropyl alcohol to others such as petroleum-ether or naphtha, as well as to supercritical fluids such as CO₂, as described above. The exact conditions and solvents applied affect the taste, color, and viscosity of the final product, but in any event, the extracting fluid should not be present in the extracted CBD oil. Many other plant components are typically co-extracted with the desired cannabinoids and other components that are present in the original plant material. To the extent that such other components are not desired, they can be removed by secondary treatment processes, such as low temperature conditioning and filtration or centrifugation. For example, the extract can be placed in a freezer (at −20 to −80° C.) for 24-48 hours, wherein components having a higher melting point such as waxes and triglycerides, as well as chlorophyll will precipitate, and they can be removed by filtration or centrifugation. This treatment can concurrently improve the taste and color of the CBD oil.

CBD oil can contain various concentrations of CBD, tetrahydrocannabinol (THC), and minor cannabinoids, mainly depending on the original plant, for example industrial hemp or cannabis variety used for extraction. Accordingly, CBD oil can contain cannabigerol (CBG) as well. CBD oil of the present invention should contain little (very low trace amounts) or none of THC. Terpenes may or may not be present in the extracted CBD oil, depending on the preparation method used because they are highly volatile and the use of elevated temperatures, such as may be applied during drying of plant materials, or during the evaporation of solvents, may result in a significant loss of terpene components. However, it is possible to capture evaporated terpenes by condensation, and reintroduce them back into the final oil or separately sourced terpenes can be added to CBD oil not otherwise containing terpenes or containing terpenes in lesser quantities than desired. Additional compositional components may be added to further adjust CBD oil and overall composition properties such as color, viscosity, taste, shelf-life stability as well as functional characteristics of the inventive composition comprising hydrolyzed collagen and CBD.

Additionally desirable components typically resulting from hemp extraction or otherwise suitable for incorporation in compositions of the present invention include terpenes and essential oils.

As discussed above, CBD oil extracted from industrial hemp plants, including from the stalks and seeds is commonly referred to as full spectrum CBD oil, which means that significant components of the whole plant remain intact. Thus, full spectrum CBD oil contains not only CBD, but also fatty acids, waxes, chlorophyll, vitamins including vitamins A, B complex vitamins such as riboflavin, thiamine and niacin, C, E, and beta-carotene, minerals including magnesium, iron, zinc, potassium, phosphorous and calcium, fatty acids, terpenes, flavonoids, and other materials that are concurrently extracted from the hemp plant by the extraction process. It may also contain approximately 100 or more other cannabinoids originally present in industrial hemp, some of which include cannabinol (CBN), cannabicyclol (CBL), tetrahydrocannabivarin (THCV), cannabigerol (CBG), cannabidiolic acid (CBDa) and cannabichromevarinic acid (CBCVA). In the original hemp plant, CBDa can be present in a greater amount than CBD; heating CBD oil to effect decarboxylation can convert CBDa to CBD.

Full spectrum CBD oil is also a source of all 20 amino acids, including the nine essential amino acids that must be provided through the diet. The two primary essential fatty acids, omega 3 and omega 6, are ideally consumed at a ratio of around 3:1. Unfortunately, in the typical diet, that ratio is close to 25:1, whereas full-spectrum CBD oil offers these two essential fatty acids in the optimal 3:1 ratio.

A potential advantage to using full spectrum CBD oil compared to CBD isolate is that the presence in full spectrum CBD oil of cannabinoids, terepenes, vitamins, and the other naturally present components discussed above can interact synergistically to produce effects in what has become known as the entourage effect. Without intending to be bound by theory, it is believed that CBD and other cannabinoids and natural components work together synergistically to improve the absorption and more effectively influence multiple targets throughout the body. It has been reported for example that full spectrum CBD oil exhibited anticonvulsant properties, whereas CBD isolate did not.

Terpene Isolates

Terpenes or terpene isolates are fragrant oils, sometimes referred to as essential oils, that are found in cannabis and hemp plants. Terpenes are not unique to cannabis and hemp, but are also found in other plants, including fruits, pine trees, and herbs. Numerous terpenes have been identified in the cannabis plant family including in cannabis and hemp.

Research has shown that terpenes bind to receptors and neurotransmitters in the brain, which can elicit various reactions and potentially interact, for example synergistically, with other compounds, including CBD itself.

Useful terpenes include terpinolene, beta-caryophyllene, limonene, myrcene, beta-pinene, trans-ocimene, alpha-pinene, alpha-terpineol, linalool and delta-3-carene. Several terpenes, including myrcene, cintronellol, and linalool, have sedation, relaxation, and calming effects attributed to them while other terpenes, such as limonene and pinene are said to boost alertness and elevate mood. Still other useful terpenes include caryophyllene, bisabolol, and humulene. Terpenes are believed capable of contributing beneficial effects to the compositions herein; terpenes in general are said to exhibit or contribute to antiseptic, anti-bacterial, antioxidant, antifungal, pain-relieving, anti-inflammatory, anti-anxiety and anti-spastic effects.

In addition to terpenes that may be present in full spectrum CBD oil, terpenes, also referred to as terpene isolates, can be added to compositions of the invention to either supplement such already present terpenes or to introduce terpenes where a limited amount or none is present, such as in CBD isolate. Embodiments of the invention include useful amounts of terpene isolates present in compositions ranging from about 0.0001 wt % to about 0.3 wt %, based on the weight of the overall composition, including CBD, hydrolyzed collagen and other optional components, if present. Additional useful terpene amounts range from low concentration of about 0.0001 or about 0.0002 wt % or about 0.0003 wt % or about 0.0004 wt % or about 0.0005 wt % or about 0.001 wt % or about 0.002 wt % or about 0.003 wt % or about 0.004 wt % or about 0.005 wt % or about 0.006 wt % or about 0.008 wt % or about 0.01 wt % or about 0.05 wt % or about 0.10 wt % or about 0.15 wt % or about 0.20 wt % to a high concentration of about 0.0002 wt % or about 0.0003 wt % or about 0.0004 wt % or about 0.0005 wt % or about 0.001 wt % or about 0.005 wt % or about 0.01 wt % or about 0.05 wt % or about 0.10 wt % or about 0.15 wt % or about 0.20 wt % or about 0.25 wt %. Intermediate ranges selected from a lower bound of 0.0001 wt % to an upper bound of 0.3 wt % are also suitable.

Terpene blends containing selected terpene molecules in admixture are available commercially. Terpene compositions can include, for example, terpinolene in an amount, based on the terpene composition, of about 30 to about 40 wt %, beta-caryophyllene of about 13 to about 17 wt %, limonene of about 10 to about 14 wt %, myrcene of about 9 to about 11 wt %, beta-pinene of about 6 to about 8 wt % and trans-ocimene, alpha-pinene, alpha-terpineol, linalool and delta-3-carene, each about 5 to about 6 wt % and several other terpenes in still lesser amounts.

Essential Oils

Embodiments of the present invention include compositions in which one or more essential oils are included. An essential oil is a concentrated hydrophobic liquid containing volatile, in other words, easily evaporated at normal temperatures, aroma compounds which are obtained from plants. Essential oils are also known as volatile oils, ethereal oils, aetherolea, or simply as the oil of the plant from which they were extracted, such as lavender oil or oil of clove. An essential oil is “essential” in the sense that it contains the “essence of” the plant's fragrance, in other words, the characteristic fragrance of the plant from which it is derived in contrast to the term essential as meaning indispensable when applied in connection with terms such as essential amino acid or essential fatty acid because they are nutritionally required, for example, by a particular living animal. Essential oils typically evaporate completely without leaving a stain or residue.

Essential oils are generally extracted by distillation, often by using steam. Other extraction processes include expression, solvent extraction, absolute oil extraction, resin tapping, wax embedding, and cold pressing. Essential oils are available commercially from various sources.

One or more essential oils can be included in compositions of the present invention either in the form of the essential oil or the specific chemical compound of which the essential oils are composed, for example, methyl salicylate instead of oil of wintergreen. Essential oils are typically oil soluble and thus are compatible with full spectrum CBD although essential oils can be included in embodiments of compositions of the present invention just as CBD itself is included.

Essential oils such as lavender, peppermint, tea tree oil, patchouli, and eucalyptus are obtained from distillation, typically of raw plant material, consisting of the flowers, leaves, wood, bark, roots, seeds, or peel, is put into an alembic or other suitable distillation apparatus over water. As the water is heated, the steam passes through the plant material, vaporizing the volatile compounds. The vapors flow through a coil, where they condense back to liquid, which is then collected in the receiving vessel. Most oils are distilled in a single process although fractional distillation can also be used.

Citrus peel oils including lemon and orange oils, are typically expressed mechanically or cold pressed. On the other hand, most flowers contain too little volatile oil to undergo expression, but their chemical components are too delicate and easily denatured by the high heat used in steam distillation. Instead, a solvent such as hexane or supercritical carbon dioxide is used to extract the oils. Extracts from hexane and other hydrophobic solvents are called concretes, which are a mixture of essential oil, waxes, resins, and other lipophilic or oil-soluble plant material readily extracted by the solvent.

Concretes contain large quantities of non-fragrant waxes and resins and thus often, another solvent, such as ethyl alcohol, is used to extract the fragrant oil from the concrete. In a typical process, the alcohol solution is chilled to −18° C. (0° F.) for more than 48 hours which causes the waxes and lipids to precipitate. The precipitates are then filtered and the ethanol is removed from the remaining solution by evaporation, vacuum purge, or both, leaving behind the absolute or essential oil itself.

Alternatively, supercritical carbon dioxide can be used as a solvent in a supercritical fluid extraction process. This method avoids petrochemical residues in the product due to the use of other solvents as described above and the loss of some “top notes” or highly volatile components when steam distillation is used. It does not yield an absolute directly. The supercritical carbon dioxide will extract both the waxes and the essential oils that make up the resulting concrete. Subsequent processing with liquid carbon dioxide, which can be achieved in the same extractor by merely lowering the extraction temperature, will separate the waxes from the essential oils. This lower temperature process prevents the decomposition and denaturing of compounds. When the extraction is complete, the pressure is reduced to ambient and the carbon dioxide reverts to a gas, leaving no residue.

Essential oils can be obtained from various components of many different plants, including the bark (cassia, cinnamon, sassafras), berries (allspice, juniper), flowers (cannabis, chamomile, clary sage, clove, hops, hyssop, jasmine, lavender, manuka, marjoram, orange, pelargonium (scented geranium), plumeria, rose, ylang-ylang), leaves (basil, bay leaf, buchu, cinnamon, common sage, eucalyptus, guava, lemon grass, melaleuca, oregano, patchouli, peppermint, pine, rosemary, spearmint, tea tree, thyme, tsuga, wintergreen), peel (bergamot, grapefruit, lemon, lime, orange, tangerine), resin (benzoin, copaiba, frankincense, labdanum, myrrh), rhizome (galangal, ginger), roots (valerian), seeds (anise, buchu, celery, cumin, flax, nutmeg oil) and woods (agarwood, camphor, cedar, rosewood, sandalwood). Oils obtained from specific plant materials are identified as “GRAS”, generally recognized as safe, by the US Food and Drug Administration, although some precautions may need to be observed for specific oils with respect to specific persons. A listing of such essential oils can be found on the Wikipedia webpage, “Essential Oil” (https://en.wikipedia.org/wiki/Essential_oil).

Preferred essential oils for use in the present invention may include bergamot, lavender, peppermint and chamomile; lavender oil is particularly preferred.

Limited amounts of one or more essential oils are useful in the present invention. Useful amounts range from about 0.0001 wt % to about 0.1 wt % based on the total weight of the composition. Additional useful amounts of essential oils range from low concentrations of about 0.0001 or about 0.0002 wt % or about 0.0003 wt % or about 0.0004 wt % or about 0.0005 wt % or about 0.001 wt % or about 0.002 wt % or about 0.003 wt % or about 0.004 wt % or about 0.005 wt % or about 0.01 wt % or about 0.02 wt % or about 0.03 wt % or about 0.05 wt % or about 0.06 wt % or about 0.075 wt % to a high concentration of about 0.0002 wt % or about 0.0003 wt % or about 0.0004 wt % or about 0.0005 wt % or about 0.0006 wt % or about 0.0007 wt % or about 0.00075 wt % or about 0.001 wt % or about 0.002 wt % or about 0.003 wt % or about 0.004 wt % or about 0.005 wt % or about 0.0075 wt % or about 0.01 wt % or about 0.02 wt % or about 0.03 wt % or about 0.04 wt % or about 0.05 wt % or about 0.075 wt % or about 0.08 wt % or about 0.1 wt %. Alternative intermediate ranges in addition to those recited and selected from a lower bound of 0.0001 wt % to an upper bound of 0.1 wt % are also suitable.

Dosing or Delivery

Useful compositions of the present invention comprise hydrolyzed collagen and cannabidiol, CBD, to which one or more optional ingredients as described herein can also be added. Particularly useful compositions comprise hydrolyzed collagen or enzymatically hydrolyzed collagen; preferred compositions comprise enzymatically hydrolyzed collagen and an effective amount of CBD in a unit dosage of 30 mL (or its approximate equivalent, 1 fluid ounce), although proportional alternative unit dosages in multiples of 30 mL are also effective, for example 10 mL, 20 mL, 40 mL, 50 mL, 60 mL, 90 mL, 120 mL and so on. In an embodiment, an effective amount of CBD comprises about 5 mg CBD in or corresponding to 72 mg full spectrum hemp oil and from about 10 grams hydrolyzed collagen, preferably enzymatically hydrolyzed collagen in said 30 mL. Alternative effective amounts comprise from about 1 mg to about 100 mg CBD, corresponding to about 14 mg to about 1440 mg full spectrum hemp oil in a 30 mL unit dose; or about 2 mg to about 90 mg CBD, corresponding to about 29 mg to about 1296 mg full spectrum hemp oil; or about 3 mg to about 80 mg CBD corresponding to about 43 mg to about 1150 mg full spectrum hemp oil; or about 4 mg to about 70 mg CBD, corresponding to about 58 mg to about 1008 mg full spectrum hemp oil; or about 5 mg to about 60 mg CBD, corresponding to about 72 mg to about 864 mg full spectrum hemp oil; or about 6 mg to about 50 mg, corresponding to about 86 mg to about 720 mg full spectrum hemp oil; or about 7 mg to about 40 mg CBD, corresponding to about 101 mg to about 576 mg full spectrum hemp oil; or about 8 mg to about 30 mg CBD, corresponding to about 115 mg to about 432 mg full spectrum hemp oil; or about 9 mg to about 20 mg CBD, corresponding to about 130 mg to about 288 mg full spectrum hemp oil.

Alternatively, effective compositions comprise from about 250 mg to about 20 g hydrolyzed collagen, preferably enzymatically hydrolyzed collagen and from about 1 mg to about 100 mg CBD; all alternative intermediate ranges for each of enzymatically hydrolyzed collagen and CBD are included in this disclosure.

In alternative embodiments with respect to the amount of hydrolyzed collagen, including enzymatically hydrolyzed collagen, for example from about 250 mg, or in an amount increasing by increments of about 50 mg above 250 mg to about 20 g; in other words about 300 mg, or about 350 mg, or about 400 mg, or about 450 mg, etc. to about 20 g.

As disclosed hereinabove, hydrolyzed collagen useful in the present invention, also referred to as enzymatically hydrolyzed collagen comprises a mixture of amino acids, including essential and non-essential amino acids. Being derived from natural collagen sources, enzymatically hydrolyzed collagen and the amounts and percentages of amino acids comprised therein correspondingly vary. However, the delivery of enzymatically hydrolyzed collagen in the amounts disclosed immediately above necessarily provides for the delivery of each of the amino acids comprised in the hydrolyzed collagen as well as additional components that may be added according to the disclosure herein. The following tables briefly summarize percentages of significant amino acid components that may be found in enzymatically hydrolyzed collagen as well as a more detailed summary of such amino acid components:

Brief Summary of Amino Acids Present in Enzymatically
Hydrolyzed Collagen

| Amino acids | Percentage |
|---|---|
| Proline and hydroxyproline | 25% |
| Glycine | 20% |
| Glutamic acid | 11% |
| Arginine | 8% |
| Alanine | 8% |
| Other essential amino acids | 16% |
| Other non-essential amino acids | 12% |

Detailed Summary of Amino Acids Typically Present in Enzymatically
Hydrolyzed Collagen

| | | | Typical |
|---|---|---|---|
| Amino Acid | Classification | Range (%)* | (%) |
| Alanine | NE | 8.00-11.00 | 8.84 |
| Arginine | CE | 7.50-9.10 | 7.75 |
| Aspartic Acid | NE | 4.50-6.95 | 5.50 |
| Cystine | CE | 0.00-0.50 | 0.02 |
| Glutamic Acid | CE | 8.90-11.50 | 9.98 |
| Glycine | CE | 19.00-30.50 | 23.19 |
| Histidine | E | 0.41-1.50 | 0.53 |
| Hydroxylysine | M | 0.50-1.50 | 0.82 |
| Hydroxyproline | M | 11.90-14.70 | 11.90 |
| Isoleucine | E | 1.20-1.90 | 1.39 |
| Leucine | E | 2.50-3.50 | 3.02 |
| Lysine | E | 2.80-5.20 | 3.76 |
| Methionine | E | 0.50-1.55 | 0.85 |
| Phenylalanine | E | 1.50-2.56 | 1.99 |
| Proline | CE | 9.30-18.00 | 12.60 |
| Serine | NE | 2.90-4.20 | 3.16 |
| Threonine | E | 1.60-3.00 | 2.02 |
| Tryptophan | E | 0.00 | 0.00 |
| Tyrosine | CE | 0.19-1.00 | 0.34 |
| Valine | E | 2.18-3.60 | 2.33 |
| Total | | — | 100.00 |

Key: E = Essential Amino Acid, NE = Non-Essential Amino Acid; CE = Conditionally Essential Amino Acid; M = Modified Amino Acid
*Ranges based on alternate material sources including bovine, porcine, marine

Therefore, referring, for example to the above summary table and to the several ranges for the amount of hydrolyzed collagen disclosed immediately above, the delivery, for example, of 10 g of enzymatically hydrolyzed collagen necessarily also delivers each of the identified amino acids in the percentages shown, for example, 2.5 g of proline and hydroxyproline, 2 g of glycine, 1.1 g glutamic acid, 0.8 g each of arginine and alanine, as well as 1.6 g of other essential amino acids not specifically identified in the summary table, but further identified in the detailed table (including histidine, isoleucine, leucine, lysine, methionine, phenylalanine, threonine, tryptophan and valine) and 1.2 g of other non-essential amino acids (although it is noted that arginine is considered an essential amino acid for children), aspartic acid, cystine, hydroxylysine, serine, and tyrosine). Additionally, as also disclosed herein, to the extent that an effective amount of the essential amino acid tryptophan is added in order to supplement the amino acids present in hydrolyzed collagen, it too will be delivered in its effective amount.

The same straightforward calculation can be carried out, in other words multiplying each of the above percentages to the useful amounts of hydrolyzed collagen disclosed above, from 250 mg to 20 g in 50 mg increments, in order to arrive at the amounts of amino acids delivered by the compositions claimed herein. Also as disclosed herein, the amounts and percentages of amino acids in any given sample of hydrolyzed collagen can vary due to natural compositional variations in the source collagen and thus the amino acid profile of hydrolyzed collagen suitable for use in the present invention can be varied by as much as ±30% by weight; more typically ±25% by weight; for example, ±20% by weight; provided that, if the amount of any particular amino acid is less than the preferred, desired or target amount, it is at least sufficient so that the composition is suitable to achieve the desired benefit of the composition. Alternatively, the amount of each amino acid present in the hydrolyzed collagen for use in the present invention can be adjusted or supplemented to the desired or preferred level.

In alternative embodiments with respect to the amount of CBD, including CBD isolate or the amount of CBD delivered in CBD oil or in full spectrum CBD oil, for example from about 1 mg or in an amount increasing by increments of 0.1 mg above 1 mg to about 100 mg; in other words, about 1.1 mg, about 1.2 mg, about 1.3 mg, etc. to about 100 mg.

As disclosed herein, full spectrum CBD or hemp oil can contain, in addition to CBD and extracted cannabinoids and terpenes, a carrier oil, such as olive oil, sunflower oil or hemp oil. Preferred, full spectrum CBD comprising olive oil is available commercially.

Compositional embodiments disclosed herein comprise those which produce or are reported to produce an anti-anxiety or anti-psychotic effect or an anxiolytic relief of anxiety-related disorders and fear or an anti-inflammatory effect in an individual who ingests a composition of the invention or to whom a composition is administered. Such compositions further produce or are reported to facilitate improvements in rest and/or in recovery from pain and improved recovery, including but not limited to recovery after physical and/or mental exertion. Improvement can be characterized as a shorter time interval for recovery by a person after physical and/or mental exertion and/or a shorter time for a person to reach a restful or so-called REM (rapid eye movement) sleep state.

A typical dose useful in the present invention is about 15 to about 60 mL of a formulated composition; preferably about 20 to about 40 mL; more preferably from about 25 to about 35 mL; for example, about 30 mL or about 1 ounce. While the dose amount can be adjusted for individual needs, a 30 mL portion is considered to be suitable for the average individual. Such a dose is typically administered or consumed about three times daily, although greater or fewer administrations are feasible, depending on individual needs. For example, if the individual is in particular need of supplementation due to personal circumstances or a physical or emotional condition, the individual or a skilled assistant or dietician can adjust the dosing amount and/or frequency as required.

Thus, ingesting a liquid form of the composition of the present invention an individual can receive effective or preferred daily amounts of enzymatically hydrolyzed collagen or protein and amino acids comprised in such enzymatically hydrolyzed collagen as well as an effective amount of CBD. For example, ingesting during a 24 hour period, about 3 to about 5 doses, for example about 3 to 4 doses, each dose about 30 mL's each, wherein each dose typically comprises about 5 to 20 grams of protein, for example about 7 to about 18 grams, or about 9 to about 17 grams, such as about 10 or 11 or 12 or 13 or 14 or 15 or 16 grams of protein. Alternatively, fewer doses of lesser volume can also be ingested or administered and still be effective, such as doses ranging from about 3 mL to about 30 mL or ⅛ fluid ounce (approximately 3.75 ml) to about 1 fluid ounce (approximately 30 ml) and including intermediate volume doses of about ¼ fluid ounce (approximately 7.5 ml) or about ½ fluid ounce (approximately 15 ml). Fewer doses of such lesser volumes can be ingested or administered during a 24 hour period and still be effective, including at least one dose or two doses or three doses or four doses or up to 20 doses and including numbers of doses between one and twenty.

In other words, a mixture of the primary, preferred components of the present invention, namely enzymatically hydrolyzed collagen and CBD or full spectrum CBD in effective amounts can be dispersed in a suitable liquid carrier, such as water, and ingested.

For example, useful dosing regimens based on a 30 mL dose of the composition of the present invention taken by mouth and having about 10 grams of the complete amino acid profile described above and including an effective amount of CBD, would be at least once per day or preferably twice per day or still more preferably three times per day. Alternatively, lesser amounts or volumes ingested fewer times per day as described above can also be effective, provided that the ingested composition comprises an effective amount of CDB in combination with the enzymatically hydrolyzed collagen.

In an embodiment, compositions of the invention comprise: 10 grams of enzymatically hydrolyzed collagen selected from at least one of bovine, porcine or marine collagen; 72 mg full spectrum cannabidiol oil comprising 5 mg cannabidiol; 0.003 wt % terpene isolate blend comprising terpinolene, beta-caryophyllene, myrcene, alpha-pinene, beta-pinene, trans-ocimene, alpha-terpineol, linalool, delta-3-carene and mixtures or combinations thereof and 0.001 wt % lavender essential oil. Thus, the lower dosage volumes referred to above (compared to 1 fluid ounce or 30 mL), namely about ⅛ fluid ounce or about ¼ fluid ounce or about ½ fluid ounce, contain respectively about 0.625 mg, or about 1.25 mg or about 2.5 mg cannabidiol. Correspondingly, the amounts of full spectrum cannabidiol oil, terpene isolate blend and lavender essential oil are adjusted proportionately for the reduced volumes.

Additional Optional Components

Flavonoids

Flavonoids are a group of phytonutrients that are responsible for providing the non-green pigments to plants, and for contributing to the taste and smell of fruits, vegetables, and herbs.

While flavonoids are found in many ordinary edible plants, like tomatoes and blueberries, it is believed that the cannabis family of plants, including hemp, also comprises flavonoids. Thus, flavonoids are expected to be present in full spectrum CBD oil and not in CBD isolate, although flavonoids can be added to CBD compositions useful herein that are based on CBD isolate. Flavonoids that are unique to cannabis are referred to as cannaflavins.

Flavonoids are among the largest nutrient families, and researchers have so far identified over 6,000 unique compounds. Flavonoids in general contribute to antioxidant and anti-inflammatory effects. The flavonoid cannaflavin-A, for example, has been shown to possess anti-inflammatory effects.

Sweetener and/or Flavoring Agent

Although enzymatically hydrolyzed collagen has a significantly better taste and odor than hydrolyzed collagen not produced by enzymatic hydrolysis, it does retain a certain amount of acridity. Optionally, in other words, if desired, in order to at least partially mask the underlying acrid taste, ordinary sugar (sucrose) or, more desirably, a sweetener other than ordinary sugar can be added to the composition. In such instances, artificial sweeteners, such as sodium saccharin or the like, are not desirable because the aftertaste of at least some types of artificial sweeteners, when combined with the acrid taste of non-enzymatically hydrolyzed collagen, would make the overall composition relatively unpalatable. In compositions of the present invention, even though a certain small amount of residual acridity may be present, the taste is capable of being readily masked by artificial sweeteners, with no serious aftertaste problem, except as may be present in the sweeteners themselves, depending on the type of sweetener used. Preferably, where a sweetener is added, sorbitol is preferred. Sorbitol not only tastes sweet itself, but it also produces a surface coating and lubricating effect thereby facilitating ingestion of the composition. Additionally, sorbitol coats the taste buds, further masking residual acrid taste, if any. If used in the present invention the sweetener can be a natural sweetener, an artificial sweetener or mixtures thereof. For example, artificial sweetener can be selected from acesulfame potassium, aspartame, neotame, saccharin, sucralose, alitame, cyclamate and mixtures thereof. On the other hand, natural sweetener can be selected from tagatose, trehalose, a dihydrochalcone, clycyrrhizin, stevioside, thaumatin, erythritol, hydrogenated starch hydolysates, isomalt, lactitol, maltitol, mannitol, sorbitol, xylitol and mixtures thereof. Preferably, the sweetener is the artificial sweetener sucralose or another artificial sweetener with little or no aftertaste. Finally, a flavor enhancer or flavoring agent also can be included in the composition to improve the palatability of the composition. Consequently, compositions useful in the present invention can be palatable, which is distinctly advantageous. Thus, compositions can be produced that are a sugarless, lipid-free, and free of carbohydrates that might otherwise effect a rapid rise of blood glucose levels. In this respect, sorbitol, unlike ordinary sugar or sucrose, only slowly affects the blood glucose

Effervescent Agents

Compositions of the present invention are amenable to incorporation of effervescent agents, particularly when the hydrolyzed collagen component is in the form of a powder or as a component comprising a carbonated beverage. Since a smaller amount of the CBD component, as well as terpenes and flavonoids, are used in the inventive composition, they can be incorporated into hydrolyzed collagen in the form of liquids. Alternatively, if CBD isolate is used, it is readily converted to a solid, e.g., crystallized and milled to a smaller particle size, and incorporated into the composition in the form of a powder. Similarly, to the extent that any of the components for the composition are available or convertible to solids, such other components can readily be mixed together with hydrolyzed collagen and CBD isolate powder to produce the desired composition. Effervescent components can be added to the composition in order to introduce effervescence when water is added, thus obtaining additional benefits available from effervescence, such as improved absorption and improved palatability or mouth feel. Alternatively, an effervescent agent can be incorporated in the composition such that effervescence is produced after ingestion of the composition, for example, in the form of a capsule or tablet.

Useful effervescent components include at least one member from (1) or from each of (1) and (2):

(1) sodium carbonate, sodium bicarbonate, sodium phosphate, sodium hydrogen phosphate, sodium dihydrogen phosphate, sodium hydroxide, potassium carbonate, potassium bicarbonate, potassium hydrogen phosphate, potassium dihydrogen phosphate, potassium hydroxide, magnesium carbonate, magnesium hydroxide, magnesium oxide, calcium carbonate, calcium oxide, and mixtures thereof; and

(2) a compound selected from the group consisting of citric acid, sodium citrate, potassium citrate, maleic acid, maleic anhydride, sodium malate, potassium malate, tartaric acid, sodium tartrate, potassium tartrate, fumaric acid, sodium fumarate, potassium fumarate, ascorbic acid, sodium ascorbate, potassium ascorbate, and mixtures thereof.

If effervescence is generated in vivo, without being limited by theory, it is postulated that the effervescent agent can in certain instances help to drive other components of the composition from the stomach into small intestine, and therefore into the blood stream. Part of the reason for this is that in the highly acidic environment of the stomach, the effervescent agent, which is typically the salt of a basic material or the mixture of a salt of a basic material and an acidic material or its salt, can react with the stomach acid to generate carbon dioxide bubbles, i.e. effervescence, which in turn can aid in tablet disintegration and movement or absorption of the other components of the composition from the stomach and into the bloodstream.

In some embodiments, the invention relates to a composition wherein the effervescent agent comprises sodium bicarbonate, sodium dihydrogen phosphate, and citric acid.

In some embodiments, the invention relates to a composition wherein the effervescent agent comprises sodium bicarbonate and sodium dihydrogen phosphate.

In some embodiments, the invention relates to a composition wherein the effervescent agent comprises sodium bicarbonate, sodium dihydrogen phosphate monohydrate (also known as sodium phosphate monobasic monohydrate), and anhydrous citric acid.

In some embodiments, the invention relates to a composition wherein the effervescent agent comprises sodium bicarbonate and sodium dihydrogen phosphate monohydrate.

Preferably, the effervescent agent comprises a mixture of sodium bicarbonate, sodium dihydrogen phosphate and citric acid. Furthermore, effervescent agent components, including (1) and/or (2) from above, are present in the composition in an amount of from about 0.1% to about 50% by weight of an effervescent agent, as compared to the total weight of the composition.

In some embodiments, particularly in tablet or capsule delivery of inventive compositions, the effervescent agent is present in the compositions described herein in the amount from about 0.1 to about 1500 mg. In some embodiments, the effervescent agent is present in the compositions described herein in the amount from about 100 mg to about 750 mg. In some embodiments, the effervescent agent is present in the compositions described herein in the amount from about 250 mg to about 500 mg. In some embodiments, the effervescent agent is present in the compositions described herein in the amount from about 0.1 to about 10 mg, from about 10 mg to about 20 mg, from about 20 mg to about 50 mg, from about 50 mg to about 100 mg, from about 100 mg to about 200 mg, from about 200 mg to about 500 mg, from about 500 mg to about 1000 mg or from about 1000 mg to about 1500 mg. In some embodiments, the effervescent agent is present in the compositions described herein in the amount of about 5 mg, about 10 mg, about 15 mg, about 20 mg, 25 mg, about 50 mg, about 75 mg, about 100 mg, about 125 mg, about 150 mg, about 175 mg, about 200 mg, about 225 mg, about 250 mg, about 275 mg, about 300 mg, about 325, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1025 mg, about 1050, mg, about 1075 mg, about 1100 mg, about 1125 mg, about 1150 mg, about 1175 mg, about 1200 mg, about 1225 mg, about 1250 mg, about 1275 mg, about 1300 mg, about 1325 mg, about 1350 mg, about 1375 mg, about 1400 mg, about 1425 mg, about 1450 mg, about 1475 mg, and about 1500 mg.

Emulsifiers

As described herein, CBD useful in the present invention can be in the form of an oil, such as full spectrum CBD or hemp oil, or a powder, such as CBD isolate. On the other hand hydrolyzed collagen, or more preferably enzymatically hydrolyzed collagen (EHC), is typically a water soluble solid composition. Additionally, as described above, compared to CBD, EHC is used in a greater amount in compositions comprising CBD and EHC. Thus, useful compositions can be prepared by dispersing or mixing CBD powder or oil in HC or EHC powder or in an aqueous solution or dispersion of EHC along with other, optional and/or desirable components such as one or more terpenes essential oils, flavonoids, etc. Dispersion of CBD, especially CBD oil and other oleaginous components in an aqueous HC or EHC composition need not exhibit long term stability since the resulting composition can be ingested or administered shortly after it is prepared. Thus, a dispersed mixture can be shaken, for example shaken vigorously and ingested shortly thereafter. However, for improved stability the resulting mixture or dispersion can also be prepared by incorporating an emulsifier or surfactant so as to form a more stable oil in water emulsion.

The term “emulsion” refers to a mixture or dispersion of at least two immiscible substances, the resulting liquids in the present invention, namely CBD oil or powder and EHC dispersed or dissolved in water, in which one substance, the dispersed phase, is dispersed in the other substance, the continuous phase. An emulsion is stabilized, in other words the dispersed phase remains dispersed during the relevant time period, such as during storage and/or immediately prior to and during use, with the assistance of one or more substances commonly referred to as emulsifiers. An emulsion can be a water-in-oil emulsion or an oil-in-water emulsion depending on such variables as the amount of oil (as well as type of oil) and water present, the conditions used to prepare the emulsion, the emulsifier type and amount, the temperature and combinations of such variables. The particle size or droplet size of the dispersed phase can vary over a significant range and the emulsion can remain stable, but its properties and suitability for a specific use may vary depending on the particle size of the dispersed phase. Particle size is typically expressed in terms of mean or average size since the uniformity of the dispersed phase can also vary depending on the variables noted above. Particle size does not require that the particles are necessarily spheres and the size of the particles can be based on a major or average dimension of each particle, although in a system comprising a dispersed liquid phase in a continuous liquid phase, fluid dynamics suggest that the dispersed particles will tend to be substantially spherical.

The term “emulsifier” or “surfactant” refers to a compound or mixture of compounds that has the capacity to promote formation of an emulsion and/or substantially stabilize an emulsion, at least for the short-term, i.e., during the time of practical or commercial interest, such as during storage or during use or both. An emulsifier provides stability against significant or substantial aggregation or coalescence of the dispersed phase of an emulsion. An emulsifier is typically considered to be a surface active substance in that it is capable of interacting with the dispersed and continuous phases of an emulsion at the interface between the two. For purposes herein a “surfactant” and an “emulsifier” are considered equivalent or interchangeable terms. Furthermore, included within the scope of the generic term “surfactant” are the various types of surfactants such as nonionic, ionic or partially ionic, anionic, amphoteric, cationic and zwitterionic surfactants. To be useful in the present invention the emulsifier or surfactant also needs to be suitable for ingestion.

The terms “stability” or “stable” when used in reference to an emulsion refer to the oil phase, namely CBD remaining dispersed or substantially dispersed in the aqueous or EHC phase. In other words, substantially no phase separation occurs as indicated by visual observation after a moderate period of time, for example, 1, or 2 or 3 or 4 or up to 8 hours or overnight, or alternatively, no visual separation occurs in the emulsion for the period of time between its preparation and use, for example, by ingestion.

Since compositions of the present invention include greater amounts of hydrolyzed collagen they will also include a greater amount of water in which the collagen component is dissolved compared to oleaginous components such as CBD isolate, full spectrum CBD or hemp oil and essential oils and flavonoids, when included. Thus, the resulting composition will be understood to be an oil-in-water, O/W emulsion and useful emulsifying agents or surfactants should be suitable for maintaining the stability of an O/W emulsion.

One or more suitable emulsifiers or surfactants can be used in compositions of the invention. Such emulsifiers are selected from those capable of stabilizing oil-in-water emulsions, typically exhibiting a hydrophilic-lipophilic balance, HLB, value equal to or greater than about 8, such as 8 to 30, preferably greater than about 9, for example, about 10 to about 25; preferably about 10 to about 20, such as those having an HLB value of about 10 to about 18; such as about 9 to about 17; for example about 10 to about 15 or about 9, or 10 or 11 or 12 or 13 or 14 or 15. Suitable emulsifiers or surfactants have an HLB value selected from the group consisting of about 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5 23. 23. 5, 24, 24.5, 25, 25.5, 26, 26.5, 27, 27.5, 28, 28.5, 29, 29.5 and 30.

Particularly preferred emulsifiers or surfactants are those considered suitable for use with foods and more particularly those found in nature or derived from natural products. Thus, lecithin, extracted from soybean oil can be processed such as by partial enzymatic hydrolysis to obtain a lecithin-based emulsifier product having an HLB value equal to or greater than 10. Alternatively, by esterification of the sucrose molecule, it is possible to obtain emulsifiers with HLB values ranging from 1 up to 16 for high mono-esters. High HLB lecithin emulsifiers are available from ADM Specialty Products and Giiava Singapore Ltd.

The following tabulation provides examples of surfactants useful for purposes of the invention, although useful surfactants are not limited to those specifically identified, provided the surfactant or emulsifier is suitable for use with the components of the inventive compositions to stabilize an O/W emulsion. In other words, that the surfactant or mixture of surfactants forms a stable or substantially stable oil-in-water emulsion and does not adversely affect the desirable attributes or effects of the composition can be used.

Thus a person skilled in the art can select emulsifiers from a broad range of commercially available products in order to obtain at least one emulsifier useful with the inventive compositions for stabilizing an oil-in-water emulsion. The emulsifier is preferably approved for use in foods and/or pharmaceuticals.

Following is a list of potentially useful emulsifiers and their HLB values:

| Product Name* | Chemical Identification or Synonym | HLB |
|---|---|---|
| Triton SP-135 | | 8.0 |
| Span 20 | Sorbitan monolaurate | 8.6 |
| | PEG-block-PPG-block-PEG, Mn = 5800 | 9.5 |
| | PPG-block-PEG-block-PPG, Mn = 2700 | 9.5 |
| Brij 30 | Polyoxyethylene(4) lauryl ether | 9.7 |
| Igepal CA-520 | Polyoxyethylene(5) isooctylphenyl ether | 10.0 |
| Igepal CO-520 | Polyoxyethylene(5) nonylphenyl ether | 10.0 |
| | Polyoxyethylene sorbitol hexaoleate | 10.2 |
| Merpol SE surfactant | | 10.5 |
| | Sodium stearoyl lactylate | 10-12 |
| Tween 85 | Polyoxyethylene(20) sorbitan trioleate | 11.0 |
| | 8-Methyl-1-nonanol propoxylate-block-ethoxylate | 11.0 |
| | Polyoxyethylene sorbitan tetraoleate | 11.4 |
| Triton X-114 | Polyoxyethylene(8) isooctylphenyl ether | 12.4 |
| Brij 76 | Polyoxyethylene(10) stearyl ether | 12.4 |
| Brij 97 | Polyoxyethylene(10) oleyl ether | 12.4 |
| Merpol OJ surfactant | | 12.5 |
| Brij 56 | Polyoxyethylene(10) cetyl ether | 12.9 |
| Merpol SH surfactant | | 12.9 |
| | 2,4,7,9-Tetramethyl-5-decyne-4,7-diol ethoxylate (5 EO/OH) | 13.0 |
| Triton SP-190 | | 13.0 |
| Igepal CO-630 | Polyoxyethylene(9) nonylphenyl ether | 13.0 |
| Triton N-101 | Polyoxyethylene branched nonylphenyl ether | 13.4 |
| Triton X-100 | Polyoxyethylene(10) isooctylphenyl ether | 13.5 |
| Igepal CO-720 | Polyoxyethylene(12) nonylphenyl ether | 14.2 |
| | Polyoxyethylene(12) tridecyl ether | 14.5 |
| | Polyoxyethylene(18) tridecyl ether | 14.5 |
| Igepal CA-720 | Polyoxyethylene(12) isooctylphenyl ether | 14.6 |
| Tween 80 | Polyoxyethylene(20) sorbitan monooleate | 14.9 |
| Tween 60 | Polyoxyethylene(20) sorbitan monostearate | 15.0 |
| | PEG-block-PPG-block-PEG, Mn = 2900 | 15.0 |
| | PPG-block-PEG-block-PPG, Mn = 2000 | 15.0 |
| Brij 78 | Polyoxyethylene(20) stearyl ether | 15.3 |
| Brij 98 | Polyoxyethylene(20) oleyl ether | 15.3 |
| Merpol HCS surfactant | | 15.5 |
| Tween 40 | Polyoxyethylene(20) sorbitan monopalmitate | 15.6 |
| Brij 58 | Polyoxyethylene(20) cetyl ether | 15.7 |
| | Polyoxyethylene(20) hexadecyl ether | 15.7 |
| | Polyethylene-block-poly(ethylene glycol), Mn = 2250 | 16.0 |
| Tween 20 | Polyoxyethylene(20) sorbitan monolaurate | 16.7 |
| Brij 35 | Polyoxyethylene(23) lauryl ether | 16.9 |
| | 2,4,7,9-Tetramethyl-5-decyne-4,7-diol ethoxylate (15 EO/OH) | 17.0 |
| Igepal CO-890 | Polyoxyethylene(40) nonylphenyl ether | 17.8 |
| Triton X-405 | Polyoxyethylene(40) isooctylphenyl ether | 17.9 |
| Brij 700 | Polyoxyethylene(100) stearyl ether | 18.8 |
| Igepal CO-990 | Polyoxyethylene(100) nonylphenyl ether | 19.0 |
| Igepal DM-970 | Polyoxyethylene(150) dinonylphenyl ether | 19.0 |
| | PEG-block-PPG-block-PEG, Mn = 1900 | 20.5 |
| | PEG-block-PPG-block-PEG, Mn = 8400 | 24.0 |
| | Ethylenediamine tetrakis(PO-b-EO) tetrol, Mn = 15000 | 24.0 |
| | PEG-block-PPG-block-PEG, average Mn = ca. 14,600 | 27.0 |

*Abbreviations in the above table:
Mn = number average molecular weight; PEG = polyethylene glycol; PPG = polypropylene glycol; EO = ethylene oxide; PO = propylene oxide; HLB = hydrophilic-lipophilic balance.

In an embodiment, an oil-in-water emulsion is prepared by combining water and CBD oil, full spectrum CBD oil or CBD isolate and hydrolyzed collagen and, if desired, one or more additional or optional components described hereinabove. A relatively stable emulsion can be prepared by mixing the components under high shear conditions to form an emulsion, the mixing preferably carried out using high mechanical shear or ultrasonic energy. Various mixing devices well known in the art can be employed to facilitate formation of an emulsified 0/W compositions, for example, mixer-emulsifiers, which typically utilize a high speed rotor operating in close proximity to a stator, paddle mixers utilizing paddles having various design configurations including, for example, reverse pitch, anchor, leaf, gate, finger, double-motion, helix, etc., including batch and in-line equipment, and the like.

In an embodiment, the above-described components are mixed together in the absence of an emulsifier and the components placed in a container, water is added, and the container is shaken vigorously, substantially immediately prior to use and the resulting dispersed mixture is ingested.

In an embodiment, a container is provided, which includes hydrolyzed collagen and a CBD component, plus one or more of optional components described herein, and including water, but in the absence of an emulsifier and the container is shaken vigorously, substantially immediately prior to use and the resulting dispersed mixture is ingested.

Compositions of the invention using a lesser amount of emulsifier or an emulsifier type or mixing conditions such that the resulting aqueous mixture contains dispersed particles are within the scope of the invention and are useful for delivering components of the mixture, including hydrolyzed collagen, CBD in one or more of its various forms and one or more of the optional components described above.

In an embodiment, compositions of the invention comprise: 10 grams of enzymatically hydrolyzed bovine collagen; 72 mg full spectrum cannabidiol oil comprising 5 mg cannabidiol; 0.003 wt % terpene isolate blend comprising terpinolene, beta-caryophyllene, myrcene, alpha-pinene, beta-pinene, trans-ocimene, alpha-terpineol, linalool, delta-3-carene and mixtures or combinations thereof; and 0.001 wt % lavender essential oil.

In an embodiment, compositions of the invention comprise: about 250 mg to about 20 g of hydrolyzed collagen, about 1 mg to about 100 mg cannabidiol, about 0.0001 wt % to about 0.3 wt % of at least one terpene isolate, about 0.0001 wt % to about 0.1 wt % of at least one essential oil, about 0.1 mg to about 1500 mg of at least one effervescent agent, and at least one of a flavonoid, sweetener, flavoring agent or emulsifier.

### EXAMPLES

### Comparative Example 1 and Example 2

In each of the examples described below, the aqueous compositions are shaken vigorously before ingestion.

Comparative example (1) At three essentially evenly spaced time intervals during a first day of normal physical and mental activity a middle-aged male ingests 30 mL of an aqueous composition comprising enzymatically hydrolyzed collagen, each 30 mL comprising 10 grams of bovine collagen peptides.

Example (2) On a second day of similarly normal physical and mental activity, the same individual ingests, at approximately the same time intervals, 30 mL each of an aqueous composition comprising enzymatically hydrolyzed collagen comprising 10 grams of bovine collagen peptides, and further comprising 72 mg full spectrum cannabidiol, 0.003 wt % of a terpene blend comprising a major amount of terpinolene and 0.001 wt % lavender essential oil.

In the evening after the conditions described above for each of (1) and (2), the individual experiences a night of sleep lasting between 6 and 8 hours. Upon awakening, the individual reports reaching a deep sleep more quickly and upon waking feeling more refreshed and rested after consuming the composition described in (2) compared to that in (1) above.

Further observational experiments have been conducted which demonstrate the advantages of the compositions disclosed herein. The following experimental design and execution was employed:

Case studies involved 11 participants between the ages of 25 and 80 years and including seven males between the ages of 25 and 67 and four females between the ages of 49 and 80 years. Each individual ingested a dosage of 1 fl. oz. (equivalent to about 30 mL) of hemp extract modified collagen (hereinafter HEMC) containing hydrolyzed collagen (AminoSculpt® produced by Health Direct®) and including full-spectrum hemp extract. Each dosage provided 10 grams of hydrolyzed collagen protein, 72 mg of full-spectrum hemp extract (containing 5 mg of CBD and other cannabinoids), plus lavender essential oil and various isolated terpenes). Where referenced, unmodified, hydrolyzed collagen extract (hereinafter HC), contained 16 grams of enzymatically hydrolyzed collagen protein (AminoSculpt® produced by Health Direct®). Enzymatically hydrolyzed collagen used for both HC and HEMC included the scope of amino acids typically found in enzymatically hydrolyzed collagen according to the distribution profile and typical values summarized below, corresponding to the summary of amino acids disclosed hereinabove.

Amino Acid Distribution of Enzymatically Hydrolyzed Collagen

| | | | Typical |
|---|---|---|---|
| Amino Acid | Classification | Range (%)* | (%) |
| Alanine | NE | 8.00-11.00 | 8.84 |
| Arginine | CE | 7.50-9.10 | 7.75 |
| Aspartic Acid | NE | 4.50-6.95 | 5.50 |
| Cystine | CE | 0.00-0.50 | 0.02 |
| Glutamic Acid | CE | 8.90-11.50 | 9.98 |
| Glycine | CE | 19.00-30.50 | 23.19 |
| Histidine | E | 0.41-1.50 | 0.53 |
| Hydroxylysine | M | 0.50-1.50 | 0.82 |
| Hydroxyproline | M | 11.90-14.70 | 11.90 |
| Isoleucine | E | 1.20-1.90 | 1.39 |
| Leucine | E | 2.50-3.50 | 3.02 |
| Lysine | E | 2.80-5.20 | 3.76 |
| Methionine | E | 0.50-1.55 | 0.85 |
| Phenylalanine | E | 1.50-2.56 | 1.99 |
| Proline | CE | 9.30-18.00 | 12.60 |
| Serine | NE | 2.90-4.20 | 3.16 |
| Threonine | E | 1.60-3.00 | 2.02 |
| Tryptophan | E | 0.00 | 0.00 |
| Tyrosine | CE | 0.19-1.00 | 0.34 |
| Valine | E | 2.18-3.60 | 2.33 |
| Total | | — | 100.00 |

Key: E = Essential Amino Acid; NE = Non-Essential Amino Acid; CE = Conditionally Essential Amino Acid; M = Modified Amino Acid
*Ranges based on alternate material sources including bovine, porcine, marine

Findings

All participants experienced positive sleep benefits. Seven were previous users of CE. Of those seven: (1) six had not experienced any sleep benefits with HC before but did experience sleep benefits from HEMC; and (2) One had experienced sleep benefits with HC but experienced enhanced sleep benefits with HEMC.

Furthermore, all participants reported enhanced sleep benefits quickly, namely within 1-2 nights after beginning use, with most reporting positive effects the first night.

Although observational reporting by individuals focused on sleep, two individuals reported that they also experienced less overall pain, less soreness and enhanced recovery following physical exercise as well as reduced pain due to a pre-existing neck injury.

The following table summarizes the observations of all participants.

Observational Studies
Hemp Extract Modified Collagen (HEMC) vs. Hydrolyzed Collagen (HC)

| | | | | | | Sleep | | |
|---|---|---|---|---|---|---|---|---|
| Case | | | Reported Observations | | | Benefits | | |
| Study | | | Better Overall | | Woke Up More | With Prior | Sleep Benefits | |
| No. | Gender | Age | Sleep | Deeper Sleep | Refreshed | HC Use | HEMC vs. HC | Other Comments |
| 1 | Male | 56 | | Yes | | No | | |
| 2 | Male | 25 | Yes | Yes | | N/A | | Better recovery |
| | | | | | | | | after exercise; |
| | | | | | | | | less soreness |
| 3 | Female | 51 | See other | See other | Yes | No | | Already sleeps |
| | | | comments | comments | | | | deeply and very |
| | | | | | | | | well; with HEMC |
| | | | | | | | | wakes up more |
| | | | | | | | | rested and feels |
| | | | | | | | | better |
| 4 | Male | 67 | Yes | | | No | | |
| 5 | Female | 63 | Yes | | | No | | Two doses; |
| | | | | | | | | noticed |
| | | | | | | | | immediate |
| | | | | | | | | benefits. |
| 6 | Male | 42 | Yes | Yes | Yes | Yes | Better, deeper | Wakes more |
| | | | | | | | sleep and | refreshed. |
| | | | | | | | awoke more | Previously better |
| | | | | | | | refreshed with | sleep with HC; |
| | | | | | | | HEMC* | better overall |
| | | | | | | | | deep sleep with |
| | | | | | | | | HEMC |
| 7 | Female | 80 | Yes | | | N/A | | |
| 8 | Male | 59 | Yes | Yes | | No | | |
| 9 | Male | 63 | Yes | | | N/A | | |
| 10 | Male | 56 | Yes | Yes | | No | Yes | ** |
| 11 | Female | 49 | Yes | Yes | | No | | Also experienced |
| | | | | | | | | less pain due to a |
| | | | | | | | | prior neck injury |

Notes:
Test Compositions (per 1 fl. oz. serving or dosage):
Hydrolyzed Collagen: AminoSculpt ®: 16 grams of collagen protein.
Hemp Extract Modified Collagen: AminoSculpt ® plus full spectrum-hemp extract: provides 10 grams of collagen protein; 72 mg of full-spectrum hemp extract (which yields 5 mg of CBD and other cannabinoids), plus essential oils and various isolated terpenes.
*Case Study participant noted that prior use of HC provided better sleep and that HEMC provided better overall deep sleep, and an improved ease falling asleep.
**Prior HC use alone by this individual at levels ranging from 5-30 g did not induce any sleep benefits, whereas sleep benefits were observed in the present study with doses as low as 1/8 fl. oz., 1/4 fl. oz. and 1/2 fl. oz., in addition to the benefit at 1 fl. oz.

Results reported for Case Study No. 10 are particularly noteworthy. As stated in the footnote to the table, improved sleep benefits were also observed with doses of ⅛ fl. oz., ¼ fl. oz. and ½ fl. oz. Constituent concentrations for these alternative and effective doses are as follows:

| Dose | Hydrolyzed | Full-Spectrum | | | | | | |
|---|---|---|---|---|---|---|---|---|
| (fl. oz.)/ | Collagen | Hemp Extract | CBD | Glycine | Prol.** | Glut.** | Arg.** | Ala.** |
| (mL)* | (g) | (mg) | (mg) | (g) | (g) | (g) | (g) | (g) |
| 1/(30) | 10 | 72 | 5 | 2 | 2.5 | 1.1 | 0.8 | 0.8 |
| 1/2/(15) | 5 | 36 | 2.5 | 1 | 1.25 | 0.55 | 0.4 | 0.4 |
| 1/4/(7.5) | 2.5 | 18 | 1.25 | 0.5 | 0.625 | 0.275 | 0.2 | 0.2 |
| 1/8/(3.75) | 1.25 | 9 | 0.625 | 0.25 | 0.313 | 0.138 | 0.1 | 0.1 |

*1 fl. oz. is equivalent to 29.57 mL; mL amounts shown are rounded up and approximate
**Prol. = proline and hydroxyproline; Glut. = glutamic acid; Arg. = arginine; Ala. = alanine

Glycine amount is calculated as 20% of hydrolyzed collagen, as shown for the amino acid distribution of collagen hereinabove. It is reported that sufficiently high doses of ingested glycine can have a beneficial effect on sleep, such dosages ranging from at least 3 g to as much as 60 g per day. (See Neuropsychopharmacology (2015) 40, 1405-1416, “The Sleep-Promoting and Hypothermic Effects of Glycine are Mediated by NMDA Receptors in the Suprachiasmatic Nucleus, N Kawai et al.) In contrast and surprisingly, as noted above, significantly lower ingested amounts of hydrolyzed collagen, also delivering glycine, when present in combination with an effective amount of CBD, induce observable benefits to both sleep as well as other beneficial effects in individuals, as summarized in the table hereinabove. Thus, ingesting a combination of collagen and CDB provides a surprisingly effective result.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

Any range of numbers recited in the specification or claims, such as that representing a particular set of properties, units of measure, conditions, physical states or percentages, is intended to literally incorporate expressly herein by reference or otherwise, any number falling within such range, including any subset of numbers within any range so recited. For example, whenever a numerical range with a lower limit, R_{L}, and an upper limit R_{U}, is disclosed, any number R falling within the range is specifically disclosed. In particular, the following numbers R within the range are specifically disclosed:

R=R_{L}+k(R_{U}−R_{L}),

wherein k is a variable ranging from 1% to 100% with a 1% increment, e.g., k is 1%, 2%, 3%, 4%, 5% . . . 50%, 51%, 52% . . . 95%, 96%, 97%, 98%, 99%, or 100%. Moreover, any numerical range represented by any two values of R, as calculated above is also specifically disclosed.

Alternatively, any numerical range recited herein, includes all values from the lower value to the upper value, in increments of one unit, provided that there is a separation of at least 2 units between any lower value and any higher value. As an example, if it is stated that the amount of a component, or a value of a compositional or a physical property, such as, for example, amount of a blend component, softening temperature, melt index, etc., is between 1 and 100, it is intended that all individual values, such as, 1, 2, 3, etc., and all subranges, such as, 1 to 20, 55 to 70, 197 to 100, etc., are expressly enumerated in this specification. For values which are less than one, one unit is considered to be 0.0001, 0.001, 0.01 or 0.1, as appropriate. These are only examples of what is specifically intended, and all possible combinations of numerical values between the lowest value and the highest value enumerated, are to be considered to be expressly stated in this application, in other words, any numerical range recited herein includes any value or subrange within the stated range.

Alternatively, the recitation of a numerical range for a variable is intended to convey that the invention may be practiced with the variable equal to any of the values within that range. Thus, for a variable which is inherently discrete, the variable can be equal to any integer value within the numerical range, including the end-points of the range. Similarly, for a variable which is inherently continuous, the variable can be equal to any real value within the numerical range, including the end-points of the range. As an example, and without limitation, a variable which is described as having values between 0 and 2 can take the values 0, 1 or 2 if the variable is inherently discrete, and can take the values 0.0, 0.1, 0.01, 0.001, 0.0001, 0.00001, 1.000001 or any other real values ≥0 and ≤2 if the variable is inherently continuous.

### ALTERNATIVE EMBODIMENTS

The following enumerated paragraphs illustrate various and alternative embodiments of the present invention:

1. A composition comprising hydrolyzed collagen and cannabidiol.

2. The composition of paragraph 1 wherein the hydrolyzed collagen is enzymatically hydrolyzed collagen.

3. The composition of any one of paragraphs 1 to 2 wherein the composition comprises an effective amount of cannabidiol.

4. The composition of any one of paragraphs 1 to 3 comprising water and wherein the composition comprises a unit dosage of about 30 mL.

5. The composition of any one of paragraphs 1 to 4 comprising about 1 mg to about 100 mg cannabidiol.

6. The composition of any one of paragraphs 1 to 5 comprising about 250 mg to about 20 g of hydrolyzed collagen.

7. The composition of any one of paragraphs 1 to 6 comprising about 0.0001 wt % to about 0.3 wt % of at least one terpene isolate.

8. The composition of any one of paragraphs 1 to 7 comprising about 0.0001 wt % to about 0.1 wt % of at least one essential oil.

9. The composition of any one of paragraphs 1 to 8 comprising about 0.1 mg to about 1500 mg of at least one effervescent agent.

10. The composition of any one of paragraphs 1 to 3 wherein the hydrolyzed collagen and cannabidiol are solid powders or particulates.

11. The composition of paragraph 10 comprising about 0.1 mg to about 1500 mg of at least one effervescent agent.

12. The composition of any one of paragraphs 1 to 11 comprising an emulsifier.

13. The composition of any one of paragraphs 1 to 3 comprising about 1 mg to about 100 mg cannabidiol and about 250 mg to about 20 g of hydrolyzed collagen.

14. The composition of any one of paragraphs 1 to 13 wherein the cannabidiol is full spectrum cannabidiol oil or cannabidiol isolate.

15. The composition of any one of paragraphs 1 to 14 comprising at least one of a flavonoid, sweetener or flavoring agent.

16. The composition of paragraph 1 comprising: 10 grams of enzymatically hydrolyzed bovine collagen; 72 mg full spectrum cannabidiol oil comprising 5 mg cannabidiol; 0.003 wt % terpene isolate blend comprising terpinolene, beta-caryophyllene, myrcene, alpha-pinene, beta-pinene, trans-ocimene, alpha-terpineol, linalool, delta-3-carene and mixtures or combinations thereof; and 0.001 wt % lavender essential oil.

17. A method comprising improving rest or recovery or both rest and recovery after physical exertion or mental exertion or both physical and mental exertion or recovery from pain, and combinations thereof, of a person in need thereof comprising ingesting a composition comprising hydrolyzed collagen and cannabidiol.

18. The method of paragraph 17 wherein the hydrolyzed collagen is enzymatically hydrolyzed collagen.

19. The method of any one of paragraphs 17 to 18 wherein the composition comprises an effective amount of cannabidiol.

20. The method of any one of paragraphs 17 to 19 wherein the cannabidiol is full spectrum cannabidiol oil or cannabidiol isolate.

21. The method of any one of paragraphs 17 to 20 wherein the composition comprises at least one effervescent agent.

22. The method of any one of paragraphs 17 to 21 wherein the composition is an aqueous composition.

23. The method of paragraph 22 comprising 3 to 5 doses of about 30 mL each, which are ingested during a time period of about 24 hours.

24. The method of any one of paragraphs 17 to 23 wherein the composition comprises at least one of a terpene isolate, an essential oil, an emulsifier, a flavonoid, a sweetener, and a flavoring agent.

25. The method of paragraph 17 comprising ingesting during a 24 hour period, 3 doses of 30 mL each of an aqueous composition comprising: 10 grams of enzymatically hydrolyzed bovine collagen; 72 mg full spectrum cannabidiol oil comprising 5 mg cannabidiol; 0.003 wt % terpene isolate blend comprising terpinolene, beta-caryophyllene, myrcene, alpha-pinene, beta-pinene, trans-ocimene, alpha-terpineol, linalool, delta-3-carene and mixtures or combinations thereof; and 0.001 wt % lavender essential oil.

1.-60. (canceled)

61. A composition comprising hydrolyzed collagen and cannabidiol,
wherein:
(a) hydrolyzed collagen is enzymatically hydrolyzed collagen;
(b) cannabidiol is full spectrum cannabidiol oil.

62. The composition of claim 61 comprising water and wherein the composition comprises a unit dosage of about 3 mL to about 120 mL and about 1 mg to about 100 mg full spectrum cannabidiol.

63. The composition of claim 61 comprising about 250 mg to about 20 g of enzymatically hydrolyzed collagen.

64. The composition of claim 61 comprising about 0.0001 wt % to about 0.3 wt % of at least one terpene isolate.

65. The composition of claim 61 comprising about 0.0001 wt % to about 0.1 wt % of at least one essential oil.

66. The composition of claim 61 comprising about 0.1 mg to about 1500 mg of at least one effervescent agent.

67. A composition comprising:
(a) enzymatically hydrolyzed collagen;
(b) cannabidiol isolate; and
(c) at least one terpene isolate.

68. The composition of claim 67 comprising about 0.1 mg to about 1500 mg of at least one effervescent agent.

69. The composition of claim 62 comprising an emulsifier.

70. The composition of claim 67 comprising about 1 mg to about 100 mg cannabidiol isolate and about 250 mg to about 20 g of enzymatically hydrolyzed collagen.

71. The composition of claim 67 comprising about 0.0001 wt % to about 0.3 wt % of at least one terpene isolate.

72. The composition of claim 67 comprising a terpene isolate blend selected from terpinolene, beta-caryophyllene, myrcene, alpha-pinene, beta-pinene, trans-ocimene, alpha-terpineol, linalool, delta-3-carene and mixtures or combinations thereof.

73. The composition of claim 61 comprising about 0.0001 wt % to about 0.1 wt % of at least one essential oil.

74. The composition of claim 61 comprising: at least about 10 grams of enzymatically hydrolyzed collagen; at least about 72 mg full spectrum cannabidiol oil comprising at least about 5 mg cannabidiol; at least about 0.003 wt % terpene isolate blend selected from terpinolene, beta-caryophyllene, myrcene, alpha-pinene, beta-pinene, trans-ocimene, alpha-terpineol, linalool, delta-3-carene and mixtures or combinations thereof; and about 0.001 wt % to about 0.1 wt % lavender essential oil.

75. A method comprising improving at least one of rest, including sleep; or recovery after physical exertion or mental exertion or both physical and mental exertion; or recovery from pain; of a person in need thereof comprising ingesting a composition comprising hydrolyzed collagen and cannabidiol,
wherein:
(a) hydrolyzed collagen is enzymatically hydrolyzed collagen;
(b) cannabidiol is full spectrum cannabidiol oil or cannabidiol isolate.

76. The method of claim 75 wherein the composition comprises about 1 mg to about 100 mg cannabidiol and about 250 mg to about 20 g of enzymatically hydrolyzed collagen.

77. The method of claim 75 wherein the composition comprises at least one effervescent agent.

78. The method of claim 76, wherein the composition is aqueous and the method comprises ingesting 3 to 5 aqueous doses of about 30 mL each, which are ingested during a time period of about 24 hours.

79. The method of claim 78 comprising ingesting during a 24 hour period at least one dose up to 20 doses wherein each dose volume comprises about ⅛ fluid ounce (approximately 3.75 ml) or about ¼ fluid ounce (approximately 7.5 ml) or about ½ fluid ounce (approximately 15 ml) to about 1 fluid ounce (approximately 30 ml).

80. The method of claim 75 wherein the quality of sleep is improved for an individual in need thereof.
